(19) United States
(12) Patent Application Publication
(10) Pub. No.: US 2024/0260904 A1
(43) Pub. Date: Aug. 8, 2024

(54) ELECTRONIC DEVICE

(71) Applicant: Valeo Systèmes Thermiques, Le Mesnil-Saint-Denis (FR)

(72) Inventor: Georges De Pelsemaeker, Le Mesnil-Saint-Denis (FR)

(73) Assignee: Valeo Systèmes Thermiques, Le Mesnil-Saint-Denis (FR)

(21) Appl. No.: 18/569,155

(22) PCT Filed: Jun. 6, 2022
(86) PCT No.: PCT/EP2022/065309

§ 371 Date: Dec. 11, 2023

(30) Foreign Application Priority Data
Jun. 10, 2021 (FR) FR2106142

(51) Int. Cl. A61B 5/00

(52) U.S. Cl. CPC A61B 5/721; A61B 5/743

(57) ABSTRACT

A system for providing assistance with delivery of diagnostic information is disclosed. The system includes a device for acquiring examination data on a person, a data-processing device arranged to receive the data obtained by the acquiring device, and a displaying device arranged to display diagnostic information regarding a disease based on an analysis of the data by the data-processing device. The acquiring device includes at least a first sensor for taking physiological measurements. The acquiring device includes a plurality of non-contact sensors arranged to work without contact with the person. At least one of the non-contact sensors is arranged to acquire a response datum provided by the person. The acquiring device includes a correcting sensor different from the first sensor and arranged to acquire at least one correction datum on the person that is different from data acquired by the first sensor.

The present invention relates to a system for providing assistance with delivery of diagnostic information, in particular with a view to detecting a disease in a person, in particular a contagious disease such as COVID-19.

At the present time, potential COVID-19 patients are detected by taking their temperature, performing supplementary clinical examinations, then carrying out a COVID-19 test that still possesses a relatively low confidence level. A pulmonary scanner may be used to confirm the seriousness of the disease with a high level of accuracy.

There is a great need to be able to rapidly detect, in particular by virtue of mobile means, people potentially ill with a contagious disease, for example the disease linked to COVID-19.

One subject of the invention is thus a system for detecting a disease in a person, in particular a contagious disease such as COVID-19, this detecting system comprising:
an acquiring device for acquiring examination data on the person, this acquiring device in particular comprising at least one sensor for taking physiological measurements such as a radar, and a thermal camera for acquiring these examination data,
a data-processing device arranged to receive these examination data obtained by the acquiring device,
a displaying device arranged to display diagnostic information regarding the disease based on an analysis of said examination data, this diagnostic information potentially being representative of a level of probability that the person is suffering from the disease.

According to one of the aspects of the invention, the data-processing device comprises an artificial-intelligence unit arranged to process the examination data obtained by the acquiring device and deliver said diagnostic information.

According to one of the aspects of the invention, the data-processing device and the displaying device form part of the same apparatus, for example a computer, in particular a portable computer.

In this case, the displaying device is a screen of the portable computer and the data-processing device comprises a microprocessor of this computer.

In a variant, the displaying device is arranged to be visible to the examined person, and the displaying device, which in particular comprises a screen, is in particular remote from the data-processing device, these displaying and data-processing devices for example being connected to each other by a wireless link, for example one using a 3G, 4G or 5G communication protocol, or by the Internet, or Wi-Fi for example.

According to one of the aspects of the invention, the displaying device, and optionally also the data-processing device, are arranged so as to be located on-board a motor vehicle.

As a variant, the displaying device, and optionally also the data-processing device, are arranged to be fixedly placed, in a building or an outer courtyard for example.

According to one of the aspects of the invention, the processing device is arranged to make the diagnosis automatically, without human intervention.

Because automatic processing is employed, the invention allows rapid diagnosis and/or mass screening, making it possible to return to work or to lift a lockdown more rapidly.

According to one of the aspects of the invention, the device for acquiring examination data is arranged to make possible contactless measurements, at a safe distance, of vital signs and thermal and visible imaging on the person to be examined.

The system according to the invention thus advantageously uses fusion of contactless measurements, at a safe distance, of vital signs, and of thermal and visible imaging.

According to one of the aspects of the invention, the artificial-intelligence unit is arranged to use a diagnostic model based on artificial intelligence and fed with a reasonable number of clinical measurements.

Because it employs relatively light equipment, the invention in particular makes possible facilitated deployment of field hospitals in order to support populations.

The invention makes it possible to perform diagnoses in a mobile manner. It is easy to set up, for example using a thermal camera, a sensor for taking physiological measurements such as a radar and a laptop personal computer. The invention makes it possible for it to be set up rapidly over a whole area.

According to one of the aspects of the invention, the acquiring device comprises a radar for acquiring data relating to the vital signs of the person, a thermal camera for taking temperature measurements and delivering temperature data, and a camera operating in the visible spectrum for characterizing the tested person, and delivering data characterizing the person.

According to one of the aspects of the invention, the data-processing device is arranged to run a diagnostic algorithm based on fusion of vital data, in particular breathing rate, respiration amplitude, inhalation and exhalation time, heart rate and any arrhythmia.

According to one of the aspects of the invention, the algorithm uses temperatures measured in noteworthy regions, which are located by image processing, or indeed the oxygen level linked to the person examined.

Preferably, these noteworthy regions for temperature measurement are located inside the mouth, on the tip of the nose, on the cheeks and the palm of the hand.

According to one of the aspects of the invention, the diagnostic algorithm uses a characterization of the person, such as data relating to age, gender, clothing, height and body mass index (BMI).

According to one of the aspects of the invention, the system is arranged to acquire examination data until the diagnostic information is made available within a time period in particular of between 30 and 120 seconds.

According to one of the aspects of the invention, the system is arranged to make it possible to acquire the examination data by making measurements at a distance of 60 cm to 2 m between the acquiring device and the person. It avoids the person having to be in contact with the acquiring device.

The invention thus permits a rapid diagnosis without additional time for being referred to a doctor, for example. The diagnostic information may, where appropriate, be sent automatically to a doctor and may be saved on cloud storage.

According to one of the aspects of the invention, the data-processing device uses an algorithm to analyze the acquired examination data and, where appropriate, to sort people with the aim of detecting diseased persons, based on all of the data collected and an artificial intelligence that is initially trained on a sample in a hospital setting.

This training of the artificial intelligence may be performed by means of a set of measurements which is collected by the system, but also by means of medical monitoring of the patients. This permits improvement of the model over time.

According to one of the aspects of the invention, the measurements performed by the acquiring device may serve to subsequently refine the diagnosis made by the artificial intelligence.

By virtue of the invention, a diagnosis may be made without contact with the person, thereby limiting the risks of contamination, this being particularly advantageous, for example, in the case of a pandemic such as that linked to COVID-19.

According to one of the aspects of the invention, the examination data measured comprise at least one of the following data: temperatures measured at different points of the body of the person to be examined, a respiratory or a cardiac characteristic.

According to one of the aspects of the invention, the acquiring device is arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature.

According to one of the aspects of the invention, the noteworthy measurement points are located by artificial intelligence by means of an object identification flowchart.

According to one of the aspects of the invention, the temperature relating to a noteworthy point is obtained by averaging over time and by averaging the temperatures of a surface defined by pixels originating from an image from an infrared camera in proximity to the noteworthy point identified on the visible image by means of an object identification algorithm.

According to one of the aspects of the invention, the personal characterizations are identified by means of the red-green-blue camera (or RGB camera) or far-infrared camera (FIR camera) in addition to reading the identity of the person, by virtue of a classification system which may be trained on RGB (red-green-blue) or infrared images. Using a larger number of parameters, in particular age, gender, height, body mass index and phenotype, for example, serves to improve the diagnostic models.

According to one of the aspects of the invention, the diagnostic model, or diagnostic algorithm, which is fed with more data, such as noteworthy temperatures of the body, an ambient temperature, a class of personal characteristics and a time of day, may be arranged to use, in addition, data on the movements of the person examined in order to check whether they have come into contact with a diseased person or have passed through an at-risk area.

According to one of the aspects of the invention, the system is arranged to operate in the absence of a radar and by using the RGB cameras in order to estimate the cardiac and respiratory parameters.

According to one of the aspects of the invention, the temperature relating to a noteworthy point may be obtained by averaging over time and by averaging the temperatures of a surface defined by pixels originating from an image from a camera in proximity to the noteworthy point. The noteworthy point is, for example, defined geometrically by means of an image area called a building box, which surrounds it, for example by means of the geometrical average of the sides of the area of the image. This image area is a surface delimited by a series of points which is constructed by an object identification algorithm.

According to one of the aspects of the invention, the system lacks an RGB camera and/or does not use the surface the temperature of which is controlled. In this case, the system uses external temperature and a model of heat transfer to clothed areas or indeed only temperature differences between noteworthy points.

According to one of the aspects of the invention, the system is arranged to use fusion of contactless measurements, in particular of vital signs, and of thermal and visible imaging.

The diagnostic information comprises a class chosen from among three predetermined classes, which are “Healthy person”, “Person suspected to have the disease” and “Person highly likely to have the disease”.

The diagnostic information may also comprise an evaluation of the severity of the disease.

Another subject of the invention is a method for delivering diagnostic information with a view to detecting a disease in a person, in particular a contagious disease such as COVID-19, this method comprising the following steps:
acquiring examination data on the person, using an acquiring device in particular comprising at least one sensor for taking physiological measurements such as a radar, and a thermal camera for acquiring these examination data,
receiving these examination data obtained by the acquiring device,
processing these examination data in order to obtain diagnostic information regarding the disease based on an analysis of said examination data,
displaying diagnostic information regarding the disease based on an analysis of said examination data, this diagnostic information potentially being representative of a level of probability that the person is suffering from the disease.

The present invention may make checks possible in the public realm in general, in particular on lanes of traffic, at entrances and exits of buildings, at airport gates and in schools.

The present invention also allows individuals' health to be monitored, for example ill individuals receiving home care.

Another subject of the invention, independently or in combination with the above, is a system for providing assistance with delivery of diagnostic information, in particular with a view to detecting a disease in a person, in particular a contagious disease such as COVID-19, this detecting system comprising:
a device for acquiring examination data on the person, this acquiring device in particular comprising at least one sensor, such as a radar, for taking physiological measurements, and a thermal camera for acquiring these examination data, this acquiring device comprising a plurality of non-contact sensors arranged to work without contact with the person, at least one of the non-contact sensors being arranged to acquire a response datum provided by the person,
a data-processing device arranged to receive these data obtained by a plurality of sensors of the acquiring device,
a displaying device arranged to display diagnostic information regarding the disease based on an analysis of said data by the data-processing device, this diagnostic information in particular potentially being representative of a level of probability that the person is suffering from the disease, this level of probability in particular potentially being a score.

The invention allows, by virtue of the plurality of sensors that work without contact with the person to be examined, sufficient data to be obtained to establish a more reliable diagnosis.

According to one of the aspects of the invention, the data-processing device comprises an artificial-intelligence unit arranged to process the examination data obtained by the acquiring device and deliver said diagnostic information.

According to one of the aspects of the invention, the data-processing device and the displaying device form part of the same apparatus, for example a computer, in particular a portable computer.

According to one of the aspects of the invention, the displaying device, and optionally also the data-processing device, are arranged so as to be located on-board a motor vehicle.

According to one of the aspects of the invention, the non-contact sensor arranged to acquire a response datum provided by the person comprises a microphone arranged to receive responses provided orally by the person.

According to one of the aspects of the invention, the non-contact sensor may, as a variant of a microphone working with voice recognition, comprise means for analyzing the face of the person in order, for example, to recognize a YES or a NO, expressed by nodding for example.

As a further variant, the non-contact sensor may comprise means allowing the person to point to an answer on a screen with a finger, without touching the screen.

According to one of the aspects of the invention, the system is arranged to present a questionnaire, for example through display of the questions of the questionnaire on a display screen or for example using an audio device that reads out the questions of the questionnaire, to the person to be examined, and the acquiring device is arranged to receive the answers provided by the person to this questionnaire, the microphone for example being used to record the answers of the person.

According to one of the aspects of the invention, the system comprises a data storage unit arranged to store the questions of the questionnaire, this data storage unit for example forming part of a computer.

According to one of the aspects of the invention, the data-processing device is arranged to receive, from the acquiring device, data relating to the answers provided by the person in response to the questionnaire, and this data-processing device is arranged to process these data in combination with data provided by other sensors of the acquiring device, with a view to delivering the diagnostic information regarding the disease.

According to one of the aspects of the invention, with a view to obtaining the diagnostic information, the data-processing device is arranged to run a diagnostic algorithm based on fusion of vital data of the person—in particular a breathing rate, a respiratory amplitude, an inhalation and exhalation time, a heart rate and any arrhythmia—also with questionnaire response data.

According to one of the aspects of the invention, the system is arranged to select the questions to be asked depending on the person to be examined, and in particular on one or more available data on the person.

According to one of the aspects of the invention, the questions to be asked are medical in nature, and useful for improving the diagnosis in combination with the other data.

According to one of the aspects of the invention, the questions are chosen from the following list: Does your chest hurt? Do you have a dry cough? Do you have a sore throat? Do you have a runny nose? Are you having difficulty breathing?

According to one of the aspects of the invention, the system is arranged to perform, using one of the non-contact sensors, a measurement the result data of which are compared with an answer provided by the person in response to a question of the questionnaire.

This allows confidence in the measurement taken with the sensor to be increased, or a reliability level to be attributed.

According to one of the aspects of the invention, the system is arranged to acquire a datum relating to a context, an ambient temperature for example.

According to one of the aspects of the invention, the system is arranged to perform sorting based on a static model with a view to delivering a score associated with the diagnosis.

According to one of the aspects of the invention, the system is arranged to carry out the following steps: receiving from the person consent data indicating that she or he consents to being subjected to the examination; acquiring data characterizing the person—such as gender, age and height—and data representative of the context; acquiring data in response to questions, in particular using a microphone, and measurements of vital signs; processing the data to allow an assessment of the level of reliability of the diagnostic information; optionally asking additional questions if necessary to refine the diagnostic information; and performing sorting based on a static model, with a view to delivering a score associated with the diagnosis.

According to one of the aspects of the invention, the non-contact sensors are arranged to allow measurements to be acquired at a distance comprised between 50 cm and 2 m from the person to be examined.

Yet another subject of the invention is a terminal including the aforementioned assisting system.

This terminal may for example comprise a processing unit arranged to process the data obtained from the sensors and to deliver the diagnostic information.

This processing unit may be a computer, with or without a display on the terminal.

This terminal in particular comprises the various sensors.

This terminal is for example installable at the entrance of a building.

Yet another subject of the invention is a method for providing assistance with delivery of diagnostic information, in particular with a view to detecting a disease in a person, in particular a contagious disease such as COVID-19, this method comprising the following steps:
acquiring examination data on the person, this acquiring device in particular comprising at least one sensor, such as a radar, for taking physiological measurements, and a thermal camera for acquiring these examination data, this acquiring device comprising a plurality of non-contact sensors arranged to work without contact with the person, at least one of the non-contact sensors being arranged to acquire a response datum provided by the person, this sensor in particular being a microphone,
receiving these data obtained by the plurality of sensors of the acquiring device,
displaying diagnostic information regarding the disease based on an analysis of said data by the data-processing device, this diagnostic information in particular potentially being representative of a level of probability that the person is suffering from the disease, this level of probability in particular potentially being a score.

Yet another subject of the invention is a system for providing assistance with delivery of diagnostic information, in particular with a view to detecting a disease in a person, in particular a contagious disease such as COVID-19, this detecting system comprising:
a device for acquiring examination data on the person, this acquiring device comprising at least a first sensor, in particular a sensor for taking physiological measurements such as a radar or a thermal camera for acquiring these examination data, this acquiring device in particular comprising a plurality of non-contact sensors arranged to work without contact with the person, at least one of the non-contact sensors in particular being arranged to acquire a response datum provided by the person,
a data-processing device arranged to receive these data obtained by the acquiring device,
in particular a displaying device arranged to display diagnostic information regarding the disease based on an analysis of said data by the data-processing device, this diagnostic information in particular potentially being representative of a level of probability that the person is suffering from the disease, this level of probability in particular potentially being a score,
the acquiring device further comprising a correcting sensor different from said first sensor and arranged to acquire at least one datum on the person, this datum being a correction datum different from data acquired by the first sensor,
the data-processing device being arranged to use this correction datum to correct the examination data delivered by the first sensor.

According to one of the aspects of the invention, the correcting sensor is arranged to acquire a datum representative of a movement of the person.

According to one of the aspects of the invention, this correcting sensor comprises a camera, in particular a 3D camera, for acquiring a datum representative of a movement of the person.

This camera is in particular a camera operating on the ToF principle (ToF standing for Time of Flight) which allows a scene to be measured in 3 dimensions (3D) in real time. This type of camera is known.

As a variant, this camera is an RGB camera for example operating with a body-segmenting tool or a platform equipped with a strain gauge, on which platform the person is positioned, the person potentially standing, lying or sitting.

According to one of the aspects of the invention, the correcting sensor is arranged to acquire a datum representative of a voice of the person.

According to one of the aspects of the invention, this correcting sensor arranged to acquire a datum representative of a voice of the person comprises a microphone (33), in particular a directional microphone, or a camera.

According to one of the aspects of the invention, the acquiring device comprises both a correcting sensor arranged to acquire a datum representative of a movement of the person and a correcting sensor arranged to acquire a datum representative of a voice of the person.

According to one of the aspects of the invention, the first sensor, in particular being a sensor for taking physiological measurements, is a radar in particular arranged to acquire a datum representative of a breathing rate of the person, in particular by detecting movement of the skin of the person.

According to one of the aspects of the invention, the radar is arranged to be placed facing the person or behind the back of the person.

According to one of the aspects of the invention, the data-processing device is arranged to correct the examination data depending on the correction datum using artificial intelligence.

According to one of the aspects of the invention, the data-processing device is arranged to obtain a correction signal based on the correction data, this correction signal varying as a function of time.

According to one of the aspects of the invention, the data-processing device is arranged to deliver the examination data, which are for example related to breathing rate, after correction using the correction signal.

The invention makes it possible to take into account noise related to movements of the person and/or to her or his voice, so as to deliver reliable and accurate examination data.

Specifically, movements and utterances of the person are liable to interfere with the acquisition of data, particularly physiological data such as breathing rate.

Yet another subject of the invention is a method for providing assistance with delivery of diagnostic information, in particular with a view to detecting a disease in a person, in particular a contagious disease such as COVID-19, this method comprising the following steps:
acquiring examination data on the person, using a device for acquiring examination data on the person, this acquiring device comprising at least a first sensor, in particular a sensor for taking physiological measurements such as a radar or a thermal camera for acquiring these examination data, this acquiring device in particular comprising a plurality of non-contact sensors arranged to work without contact with the person, at least one of the non-contact sensors in particular being arranged to acquire a response datum provided by the person,
correcting these examination data using correction data, in particular acquired using a correcting sensor.

The invention has the advantage of being able to mix data in order to obtain a more reliable and/or more robust result.

In the case of evaluation of heart rate by means of a radar, this evaluation being disrupted by the voice of the person or movement of the person, the invention allows this disruption to be evaluated using a microphone or a camera that makes lip reading possible or even by means of a 4D camera or an inertial balance.

The validity of the evaluation of this heart rate thus depends on external noise level, the posture of the person or light level for example.

According to one of the aspects of the invention, the method according to the invention comprises the step of taking into account the weight of the evaluations of the parameters acquired to obtain the result.

Thus when any disruptions are of larger amplitudes, the invention should give more weight to the least disrupted parameters.

According to one of the aspects of the invention, in the case of evaluation of heart rate by means of a radar, this evaluation being disrupted by the voice of the person or movement of the person, the method comprises the step of evaluating this disruption using a microphone or a camera that makes lip reading possible or even by means of a 4D camera or an inertial balance, with a view to correcting the examination data.

The invention and its various applications will be better understood on reading the following description and on examining the figures accompanying it:

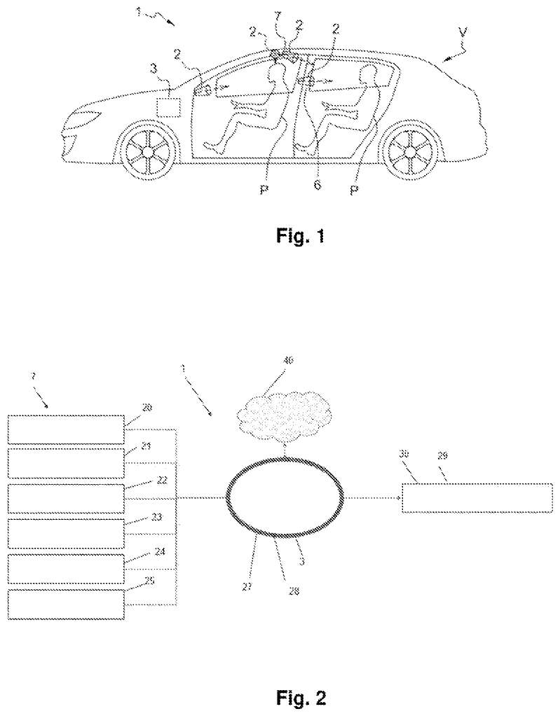

FIG. 1 schematically illustrates a system according to one non-limiting embodiment of the invention.

FIG. 2 illustrates a block diagram illustrating the steps implemented in the system of FIG. 1.

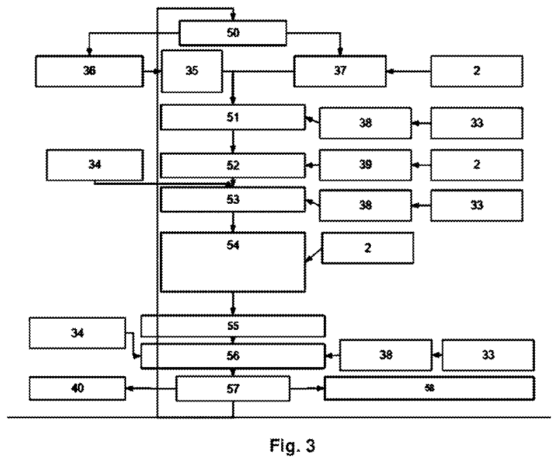

FIG. 3 illustrates a block diagram illustrating the steps implemented in a system according to another example of implementation of the invention.

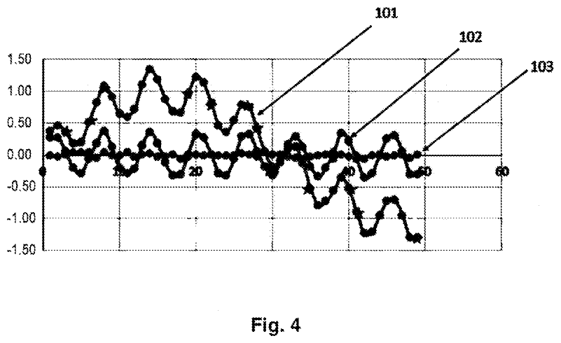

FIG. 4 shows curves illustrating the effect of the corrections obtained by virtue of the invention.

FIGS. 1 and 2 show a system 1 for providing assistance with delivery of diagnostic information, in particular with a view to detecting a disease in a person, in particular a contagious disease such as COVID-19, according to the invention.

### This System 1 Comprises:

an acquiring device 7 for acquiring examination data on the person,
a data-processing device 3 arranged to receive these examination data obtained by the acquiring device 7,
a displaying device 30 arranged to display diagnostic information regarding the disease based on an analysis of said examination data, this diagnostic information potentially being representative of a level of probability that the person is suffering from the disease.

### This System 1 in Particular Comprises:

a sensor for sensing the cardiac activity of at least one passenger, in this instance the heart rate, this sensor being a camera operating in the near infrared,
a sensor for sensing the respiratory activity, in particular in respiratory amplitude and/or frequency terms, of at least one passenger, this sensor being a camera operating in the far infrared, or thermal camera,
a radar arranged to measure vital signs of the person,
a sensor for sensing the profile characteristics of the passenger, in particular her or his gender, weight, size and age, this sensor being in this instance a red-green-blue camera, also called an RGB camera,
a card reader for reading an identity card of the person and obtaining personal data of the person.

These sensors and cameras, which form part of the acquiring device 7, have been designated by the reference sign 2 in FIG. 1. Certain sensors 2 are, for example, placed on a ceiling of the vehicle. One of the other cameras 2 is positioned in a side pillar 6 of the vehicle V.

Non-limitingly, the heart-rate and respiration sensor may be in the back of the seat or in the central console level with the thigh of the passenger.

These sensors 2 are connected in order to exchange information with a data-processing device 3 placed on the vehicle V.

The data-processing device 3 comprises an artificial-intelligence unit arranged to process the examination data obtained by the acquiring device 7 and provide said diagnostic information.

The data-processing device 3 and the displaying device 30 form part of the same apparatus, for example a computer, in particular a portable computer.

In this case, the displaying device 30 is a screen of the portable computer and the data-processing device comprises a microprocessor of this computer.

The displaying device 30 is arranged to be visible to the examined person, and the displaying device, which in particular comprises a screen, is in particular remote from the data-processing device, these displaying and data-processing devices for example being connected to each other by a wireless link, for example one using a 3G, 4G or 5G communication protocol, or by the Internet, or Wi-Fi for example.

The displaying device 30, and also the data-processing device 3, are here arranged so as to be located on-board a motor vehicle.

As a variant, the displaying device 30, and optionally also the data-processing device 3, are arranged to be fixedly placed, in a building or an outer courtyard for example.

The processing device 3 is arranged to make the diagnosis automatically, without human intervention.

The device 7 for acquiring examination data is arranged to allow contactless measurements, at a safe distance, of vital signs and thermal and visible imaging on the person to be examined.

The system according to the invention thus advantageously uses fusion of contactless measurements, at a safe distance, of vital signs, and of thermal and visible imaging.

The artificial-intelligence unit is arranged to use a diagnostic model based on artificial intelligence and fed by a reasonable number of clinical measurements.

The invention makes it possible to perform diagnoses in a mobile manner. It is easy to set up, for example using a thermal camera, a sensor for taking physiological measurements such as a radar and a laptop personal computer.

The acquiring device 7 comprises a radar for acquiring data relating to the vital signs of the person, a thermal camera for taking temperature measurements and delivering temperature data, and a camera operating in the visible spectrum for characterizing the tested person, and delivering data characterizing the person.

The data-processing device 3 is arranged to run a diagnostic algorithm based on fusion of vital data, in particular a breathing rate, a respiratory amplitude, an inhalation and exhalation time, a heart rate and any arrhythmia, and an oxygen level.

The algorithm uses temperatures measured in noteworthy areas, located by processing the image.

Preferably, these noteworthy regions for temperature measurement are located inside the mouth, on the tip of the nose, on the cheeks and the palm of the hand.

The diagnostic algorithm uses a characterization of the person, such as age, gender, clothing, height and body mass index data.

The system is arranged to acquire examination data until the diagnostic information is made available within a time period in particular of between 30 and 120 seconds.

The invention thus permits a rapid diagnosis without additional time for being referred to a doctor, for example. The diagnostic information may, where applicable, be sent automatically to a doctor and may be saved on cloud storage 40.

The data-processing device 3 uses an algorithm to analyze the acquired examination data and, where appropriate, to sort people with the aim of detecting diseased persons, based on all of the data collected and an artificial intelligence that is initially trained on a sample in a hospital setting.

The measurements performed by the acquiring device may serve to subsequently refine the diagnosis made by the artificial intelligence.

By virtue of the invention, a diagnosis may be made without contact with the person, thereby limiting the risks of contamination, this being particularly advantageous, for example, in the case of a pandemic such as that linked to COVID-19.

According to one of the aspects of the invention, the examination data measured comprise at least one of the following data: temperatures measured at different points of the body of the person to be examined, a respiratory or a cardiac characteristic.

The acquiring device 7 is arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature.

The noteworthy measurement points are located by an artificial intelligence by means of an object identification flowchart.

The personal characterizations are identified by means of the red-green-blue camera (or RGB camera) or far-infrared camera (FIR camera) in addition to reading the identity of the person, by virtue of a classification system which may be trained on RGB (red-green-blue) or infrared images. Using a larger number of parameters, in particular age, gender, height, body mass index and phenotype, for example, serves to improve the diagnostic models.

The diagnostic model, or diagnostic algorithm, which is fed with additional data, such as noteworthy temperatures of the body, an ambient temperature, a class of personal characteristics and a time of day, may be arranged to use, in addition, data on the movements of the examined person in order to check whether she or he has come into contact with a diseased person or has passed through a high-risk area.

The temperature relating to a noteworthy point may be obtained by averaging over time and by averaging the temperatures of a surface defined by pixels originating from an image from a camera in proximity to the noteworthy point. The noteworthy point is, for example, defined geometrically by means of an image area called a building box, which surrounds it. This image area is a surface delimited by a series of points which is constructed by an object identification algorithm.

The diagnostic information comprises a class chosen from among three predetermined classes, which are “Healthy person”, “Person suspected to have the disease” and “Person highly likely to have the disease”.

### The Invention Thus Implements the Following Steps:

acquiring examination data on the person using the acquiring device 7, which are the steps 20 to 25 of FIG. 2,
receiving these examination data obtained by the acquiring device,
processing these examination data in order to obtain diagnostic information regarding the disease based on an analysis of said examination data, using the data-processing device 3 (step 28),
displaying diagnostic information regarding the disease based on an analysis of said examination data, this diagnostic information potentially being representative of a level of probability that the person is suffering from the disease (step 29).

### The Steps 20 to 25 are as Follows:

the personal characterizations are identified by means of the red-green-blue cameras (or RGB cameras), which is the step 20,
acquiring the temperature of the person in the step 21 with a thermal camera of FIR type, the details of this temperature acquisition having already been described,
acquiring the breathing rate in the step 22 using the FIR camera,
acquiring the heart rate in the step 23 using the camera in the near infrared, or NIR camera,
acquiring vital signs using the radar, in the step 24,
acquiring personal data using the card reader in the step 25,
potentially oximetry by the near-infrared (NIR) camera.

The present invention also allows individuals' health to be monitored.

The examination data may, where applicable, comprise the size of the pupils and their position.

The diagnostic information is sent automatically to remote cloud storage.

Likewise, information that is useful to the diagnostic model, or diagnostic algorithm, may be received from the remote cloud.

Potentially two cameras, one NIR and one FIR, are used. The FIR camera is used to measure temperature and breathing-related characteristics and the NIR camera is used to perform the oximetry and to measure heart rate.

The acquiring device is in particular arranged to acquire examination data comprising an external temperature, a temperature measured on a cheek of the person, a temperature measured on the tip of the nose of the person, and also, where applicable, a maximum temperature of the face and a temperature of an item of clothing or of a surface at a controlled reference temperature, and, where applicable, a tidal volume, tremors and the blood oxygen level.

FIG. 3 shows another example of implementation of the invention.

In this example, the assisting system 1 comprises, as in the example described above:
a device 7 for acquiring examination data on the person, this acquiring device in particular comprising at least one sensor, such as a radar, for taking physiological measurements, and a thermal camera for acquiring these examination data, this acquiring device comprising a plurality of non-contact sensors 2 arranged to work without contact with the person, at least one of the non-contact sensors being arranged to acquire a response datum provided by the person,
a data-processing device 3 arranged to receive these data obtained by a plurality of sensors of the acquiring device,
a displaying device 30 arranged to display diagnostic information regarding the disease based on an analysis of said data by the data-processing device, this diagnostic information in particular potentially being representative of a level of probability that the person is suffering from the disease, this level of probability in particular potentially being a score.

The one of the non-contact sensors 2 arranged to acquire a response datum provided by the person comprises a microphone arranged to receive responses provided orally by the person.

The system 1 is arranged to present a questionnaire, for example through display of the questions of the questionnaire on a display screen or for example using an audio device that reads out the questions of the questionnaire, to the person to be examined, and the acquiring device is arranged to receive the answers provided by the person to this questionnaire, the microphone 33 here being used to record the answers of the person.

The system 1 comprises a data-storage unit arranged to store the questions of the questionnaire 34, this data-storage unit for example forming part of a computer.

The data-processing device 3 is arranged to receive, from the acquiring device, data relating to the answers provided by the person in response to the questionnaire, and this data-processing device is arranged to process these data in combination with data provided by other sensors of the acquiring device, with a view to delivering the diagnostic information regarding the disease.

With a view to obtaining the diagnostic information, the data-processing device 3 is arranged to run a diagnostic algorithm based on fusion of vital data of the person—in particular a breathing rate, a respiratory amplitude, an inhalation and exhalation time, a heart rate and any arrhythmia—also with questionnaire response data.

The system is arranged to select the questions to be asked depending on the person to be examined, and in particular on one or more available data on the person.

The questions to be asked are medical in nature, and useful for improving the diagnosis in combination with the other data.

The system 1 is arranged to perform, using one of the non-contact sensors 2, a measurement the result data of which are compared with an answer provided by the person in response to a question of the questionnaire.

Various steps of implementation of the invention will now be described with reference to FIG. 3.

Step 50 corresponds to the beginning of the method for detecting a disease in a person.

A step 51 of entry of data by the person to be examined follows.

In this step 51, the person for example enters data regarding herself or himself, for example her or his identity, and then optionally gives her or his consent or permission to continue with the diagnostic method.

This step 51 may be carried out by the person via a touch screen 35 coupled to a UV-based disinfecting device 36. A microphone 33 may be used to record the answers of the person to the questions. A voice-recognition device 38 is coupled to the microphone 33, so as to allow data captured by the microphone 33 to be input into the data-processing unit 3.

Context data 37, for example an ambient temperature, may be acquired by a sensor 2.

In step 52, characteristics related to the person are determined by virtue of one or more sensors 2 via a system 39 for recognizing images of objects, as described in the preceding example.

Additional questions are asked at the end of step 52 to clarify the clinical status of the person.

The questions asked in this question/answer step 53 are for example chosen from the following list: Does your chest hurt? Do you have a dry cough? Do you have a sore throat? Do you have a runny nose? Are you having difficulty breathing?

The answers may be provided by the person via the microphone 33.

In the following step 54, the sensors 2 acquire data relating to the vital signs of the person, as described in the preceding example.

Next, in step 55, the artificial intelligence delivers a preliminary score representative of the obtained diagnosis, on the basis of the data entered beforehand.

In step 56, additional questions are asked to the person, and the obtained answers are used to refine the preliminary score obtained in step 55.

A final score is given in step 57 by the artificial intelligence, which score is stored in the data-storage space 40.

In step 58, the final score is displayed for the person's information.

Another embodiment of the invention will now be described with reference to FIG. 4.

In this example, the system 1 for providing assistance with delivery of diagnostic information, which is slightly different from the one described above, comprises:
a device 7 for acquiring examination data on the person, this acquiring device comprising at least a first sensor, here a sensor for taking physiological measurements that is a radar for acquiring these examination data,
a data-processing device 3 arranged to receive these data obtained by the acquiring device,
a displaying device 30 arranged to display diagnostic information regarding the disease based on an analysis of said data by the data-processing device, this diagnostic information in particular potentially being representative of a level of probability that the person is suffering from the disease, this level of probability in particular potentially being a score,
the acquiring device 7 further comprising a correcting sensor 33 different from said first sensor, namely the radar, and arranged to acquire at least one datum on the person, this datum being a correction datum different from data acquired by the first sensor,
the data-processing device being arranged to use this correction datum to correct the examination data delivered by the first sensor.

This correcting sensor 33 is arranged to acquire a datum representative of a voice of the person.

This correcting sensor 33 arranged to acquire a datum representative of a voice of the person comprises a microphone 33, in particular a directional microphone, or a camera.

Provision may be made for the acquiring device 7 to comprise both a correcting sensor arranged to acquire a datum representative of a movement of the person and a correcting sensor arranged to acquire a datum representative of a voice of the person.

This correcting sensor for acquiring a datum representative of a movement of the person comprises a camera, in particular a 3D camera, for acquiring a datum representative of a movement of the person.

This camera is in particular a camera operating on the ToF principle (ToF standing for Time of Flight) which allows a scene to be measured in 3 dimensions (3D) in real time. This type of camera is known.

The radar is in particular arranged to acquire a datum representative of a breathing rate of the person, in particular by detecting movement of the skin of the person.

The radar is arranged to be placed facing the person or behind the back of the person.

Of course, sensors other than the radar may be used, for example a near-infrared camera.

The data-processing device 3 is arranged to correct the examination data depending on the correction datum using artificial intelligence.

The data-processing device 3 is arranged to obtain a correction signal based on the correction data, this correction signal varying as a function of time.

The data-processing device 3 is arranged to deliver the examination data, which are for example related to breathing rate, after correction using the correction signal.

The invention makes it possible to take into account noise related to movements of the person and/or to her or his voice, so as to deliver reliable and accurate examination data.

Specifically, movements and utterances of the person are liable to interfere with the acquisition of data, particularly physiological data such as breathing rate.

This may for example be seen from the curves in FIG. 4.

Curve 101 illustrates the amplitude of the error, over time, in a measurement of breathing rate on the person, this error being induced by movements of the person during the measurement and by the fact that she or he spoke during the measurement.

This error amplitude of curve 101 is relatively large.

Curve 102 shows the amplitude of the error, over time, in a measurement of breathing rate on the person, when a correction taking into account the person's movements as detected by the motion-detecting camera was taken into account.

The amplitude of curve 102 is reduced compared to curve 101.

Lastly, curve 103 illustrates the amplitude of the error, over time, in a measurement of the person's breathing rate, when corrections related to both the person's movements and speech were taken into account.

This curve 103 is close to zero by virtue of the corrections made.

The system of the invention thus makes it possible to implement the method comprising the following steps:
acquiring examination data on the person, using a device for acquiring examination data on the person, this acquiring device comprising at least a first sensor, in particular a sensor for taking physiological measurements such as a radar or a thermal camera for acquiring these examination data, this acquiring device in particular comprising a plurality of non-contact sensors arranged to work without contact with the person, at least one of the non-contact sensors in particular being arranged to acquire a response datum provided by the person,
correcting these examination data using correction data, in particular acquired using a correcting sensor.

1. A system for providing assistance with delivery of diagnostic information, with a view to detecting a disease in a person, the system comprising:
a device for acquiring examination data on a person,
wherein the acquiring device comprises at least a first sensor for taking physiological measurements
wherein the acquiring device comprises a plurality of non-contact sensors arranged to work without contact with the person,
wherein at least one of the non-contact sensors is arranged to acquire a response datum provided by the person;
a data-processing device arranged to receive the data obtained by the acquiring device; and
a displaying device arranged to display diagnostic information regarding the disease based on an analysis of the data by the data-processing device, wherein the diagnostic information is a score representing a probability that the person is suffering from the disease;
wherein the acquiring device further comprises a correcting sensor different from the first sensor and arranged to acquire at least one correction datum on the person,
wherein the correction datum is different from data acquired by the first sensor,
wherein the data-processing device is arranged to use the correction datum to correct the examination data delivered by the first sensor.

2. The system as claimed in claim 1,
wherein the correcting sensor is arranged to acquire a datum representative of a movement of the person.

3. The system as claimed in claim 2,
wherein the correcting sensor comprises a 3D camera, for acquiring a datum representative of a movement of the person.

4. The system as claimed in claim 1,
wherein the correcting sensor is arranged to acquire a datum representative of a voice of the person.

5. The system as claimed in claim 4,
wherein the correcting sensor comprises a directional microphone, or a camera configured to acquire a datum representative of a voice of the person.

6. The system as claimed in claim 1,
wherein the acquiring device comprises both a correcting sensor arranged to acquire a datum representative of a movement of the person, and a correcting sensor arranged to acquire a datum representative of a voice of the person.

7. The system as claimed in claim 1,
wherein the first sensor is a radar arranged to acquire a datum representative of a breathing rate of the person, by detecting movement of the skin of the person.

8. The system as claimed in claim 1,
wherein the data-processing device is arranged to correct the examination data depending on the correction datum using artificial intelligence.

9. The system as claimed in claim 1,
wherein the data-processing device is arranged to obtain a correction signal based on the correction data,
wherein the correction signal varies as a function of time.

10. The system as claimed in claim 1,
wherein the data-processing device is arranged to deliver the examination data after correction using the correction signal.

11. A method for providing assistance with delivery of diagnostic information, with a view to detecting a disease in a person the method comprising:
acquiring examination data on the person, using an acquiring device for acquiring examination data on the person,
wherein the acquiring device comprises at least a first sensor for taking physiological measurements,
wherein the acquiring device comprises a plurality of non-contact sensors arranged to work without contact with the person,
wherein at least one of the non-contact sensors is arranged to acquire a response datum provided by the person; and
correcting the examination data using correction data acquired using a correcting sensor.

12. The method as claimed in claim 11,
wherein the person's heart rate is evaluated by a radar,
wherein if the evaluation is disrupted by the voice of the person or movement of the person, the method comprises evaluating disruption using a sensor to correct the examination data.
